(19) United States
(12) Patent Application Publication
(10) Pub. No.: US 2009/0176102 A1
(43) Pub. Date: Jul. 9, 2009

(54) Case with fragrance, electronic device, and method for forming coating layer on the case

(76) Inventors: Tzu-Wei Lin, Taipei City (TW); Chen-Ming Sun, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/333,404

(22) Filed: Dec. 12, 2008

(30) Foreign Application Priority Data
Jan. 4, 2008 (TW) 097100299

(51) Int. Cl. B32B 27/38; A61K 8/02; B05D 1/02; B05D 1/18; B05D 3/02; B32B 27/00; B32B 27/40

(52) U.S. Cl. 428/413; 512/4; 427/427.4; 427/430.1; 427/385.5; 428/457; 428/423.1

(57) ABSTRACT

A case with fragrance and a method for forming a coating layer on the case are disclosed. The case comprises a cover and coating materials, wherein the coating materials are formed on the cover; and the coating materials comprise fragrant materials capable of releasing fragrance. The method for forming a coating layer on the case comprises the following steps: providing coating materials, the coating materials comprising a base coating and fragrant materials; stirring the coating materials, so that the fragrant materials are dissolved in the base coating; and applying the coating materials on the cover by spraying coating or dipping coating.

### BACKGROUND OF THE INVENTION

1. Field of the Invention

The present invention relates to a case, an electronic device, and a method for forming a coating layer; more particularly, the present invention relates to a case with fragrance, an electronic device having a case with fragrance, and a method for forming a coating layer on a case.

2. Description of the Related Art

For a general electronic device (such as a laptop computer or a mobile phone), in addition to functionality requirements, most manufacturers place importance on the design of its appearance. However, some senses other than sight, such as touch and smell, are currently gaining popularity.

In prior arts, some manufacturers mix fragrance with rubber or silicone materials and apply injection-molding techniques to add fragrance to products (such as toys). However, not all kinds of fragrance can be easily mixed with rubber or silicone materials.

Therefore, there is a need to provide a case with fragrance, an electronic device having a case with fragrance, and a method for forming a coating layer on the case, to mitigate and/or obviate the aforementioned problems.

### SUMMARY OF THE INVENTION

It is an object of the present invention to provide a case with fragrance.

It is another object of the present invention to provide an electronic device having a case with fragrance.

It is yet another object of the present invention to provide a method for forming a coating layer on a case.

The case with fragrance of the present invention comprises a cover and a coating layer. The coating layer is applied to the cover. The coating layer comprises fragrant materials for releasing fragrance.

The electronic device of the present invention comprises a case with fragrance. The case with fragrance comprises a cover and a coating layer. The coating layer is formed on the cover. The coating layer comprises fragrant materials for releasing fragrance.

The method for forming a coating layer on a case of the present invention comprises the following steps: providing coating materials, wherein the coating materials comprise base coating materials and fragrant materials; stirring the coating materials, such that the fragrant materials are evenly dissolved in the base coating materials; and applying the coating materials to the case by means of a spraying coating technique or a dipping coating technique.

Other advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 is a schematic drawing of a first embodiment of an electronic device of the present invention.

FIG. 2 is a schematic drawing of the first embodiment of a case of the present invention.

FIG. 3 is a schematic drawing of a second embodiment of an electronic device of the present invention.

FIG. 4 is a schematic drawing of the second embodiment of a case of the present invention.

FIG. 5 is a flowchart of a method for forming a coating layer on a case of the present invention.

### DETAILED DESCRIPTION OF THE PREFERRED EMBODIMENT

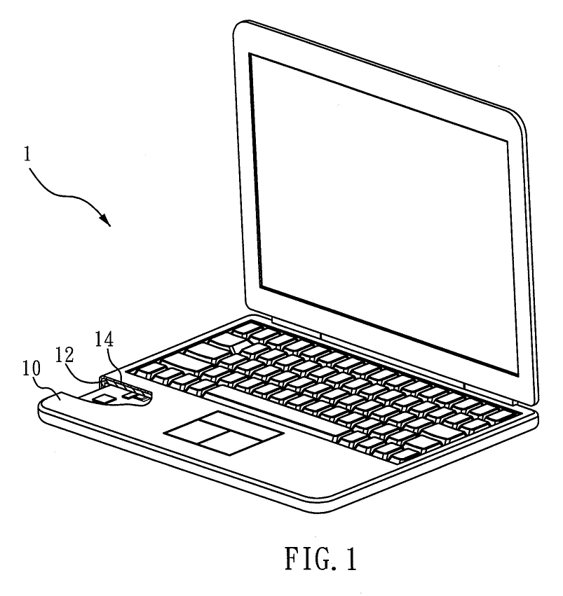

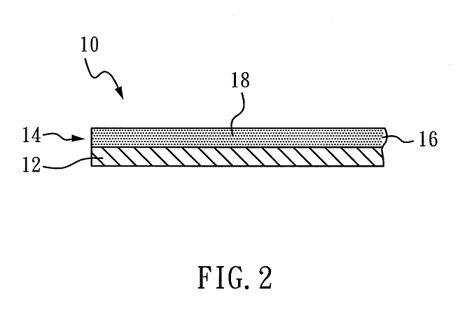

Please refer to both FIG. 1 and FIG. 2. FIG. 1 is a schematic drawing of a first embodiment of an electronic device of the present invention. FIG. 2 is a schematic drawing of the first embodiment of a case of the present invention. An electronic device 1 comprises a case 10 with fragrance. The case 10 comprises a cover 12 and a coating layer 14. The coating layer 14 is formed on the cover 12.

The cover 12 is a cover of an electronic product. For example, the cover 12 can be a cover of an electronic product such as a laptop computer, a mobile phone, a hard disk drive, or a universal serial bus (USB) drive. The raw material of the cover 12 can be plastic, metal, leather, wood, synthetic material, or any other materials to which coating materials can be applied.

In this embodiment, the cover 12 is located in the outside of the electronic product. If the electronic product is a laptop computer, the ‘cover’ can be a case, a touch pad, a monitor, a keyboard, or a battery case. Please note that the cover can also be a cover of an internal component of the electronic product, such as a cover of a hard disk drive, an optical disk drive, or a heat sink.

In this embodiment, the coating layer 14 comprises a base layer 16 and fragrant materials 18. The fragrant materials 18 are evenly applied to the base layer 16. The base layer 16 can be resin coatings or other coating materials, and the coating materials can be transparent or colored according to different requirements. The base layer 16 can be metallic paint, epoxy resin, polyurethane resin, or UV-curing acrylic resin.

The fragrant materials 18 can release fragrance. In this embodiment, the fragrant materials 18 can release plant fragrance (such as lavender fragrance or tulip fragrance) or certain fragrance representing a brand image. The fragrant materials 18 are in the form capsules (or in the form of powder if particles are fine) or liquid, such that the fragrant materials 18 can be evenly dissolved in the base layer 16 by means of stirring.

The coating materials are formed on the cover 12 by means of a spraying coating technique or a dipping coating technique. The coating materials are originally in the form of liquid, and the coating materials will become a solid coating layer 14 after drying out. The color of the coating materials can be varied according to different requirements. The coating materials can be transparent, silver, or other colors.

In this embodiment, the coating materials are formed on the outside of the cover 12, which is the side a user can directly see. Therefore, the coating materials can naturally release fragrance, and the coating layer 14 can enhance the effect of releasing fragrance through contact with the user's hand. Please note that the location where the coating layer 14 is formed on the cover 12 is not limited to the above description. The coating layer 14 can be formed on the inside of the cover 12, which is the side the user cannot directly see.

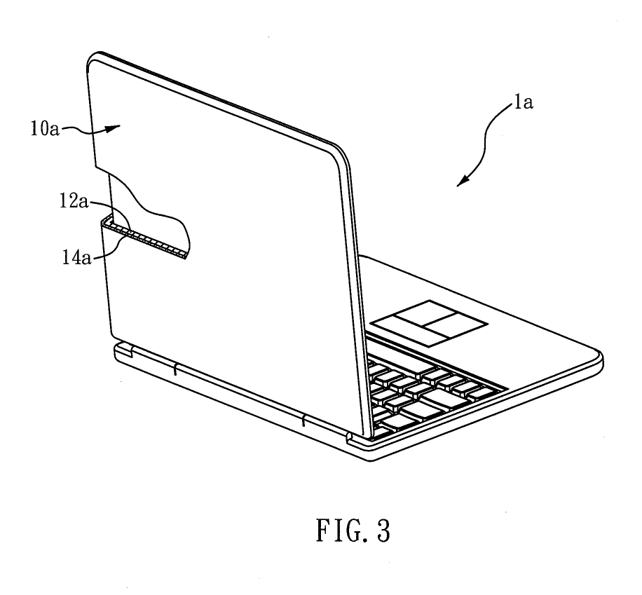

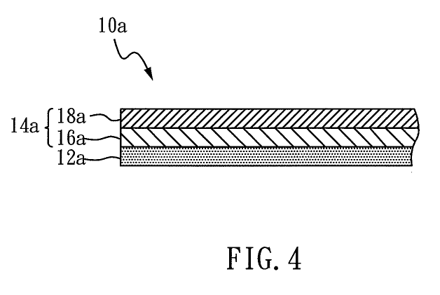

Please refer to both FIG. 3 and FIG. 4. FIG. 3 is a schematic drawing of a second embodiment of an electronic device of the present invention. FIG. 4 is a schematic drawing of the second embodiment of a case of the present invention. An electronic device 1a of the present invention comprises a case 10a with fragrance. The case 10a comprises a cover 12a and a coating layer 14a.

In the above first embodiment, the fragrant materials 18 are evenly applied to the base layer 16 (as shown in FIG. 2). While in this embodiment, fragrant materials 18a and a base layer 16a are respectively independent coating layers (as shown in FIG. 4). The coating layer 14a comprises both the base layer 16a and the fragrant materials 18a. The fragrant materials 18a are applied to the base layer 16a by means of a spraying coating technique or a dipping coating technique. In other words, the base layer 16a is located between the cover 12a and the fragrant materials 18a. In this embodiment, the fragrant materials 18a are in the form of liquid.

The base layer 16a can be a primer paint layer, an inter-coat layer, a finish paint layer, or any combination thereof. The base layer 16a usually has protective, wear-resisting or artistic functions. If the base layer 16a is only the finish paint layer, the finish paint layer is formed on the cover 12a, and the fragrant materials 18a are applied to the finish paint layer. If the base layer 16a includes the primer paint layer, the inter-coat layer and the finish paint layer, the cover 12a is sequentially formed with the primer paint layer, the inter-coat layer, the finish paint layer, and the fragrant materials 18a.

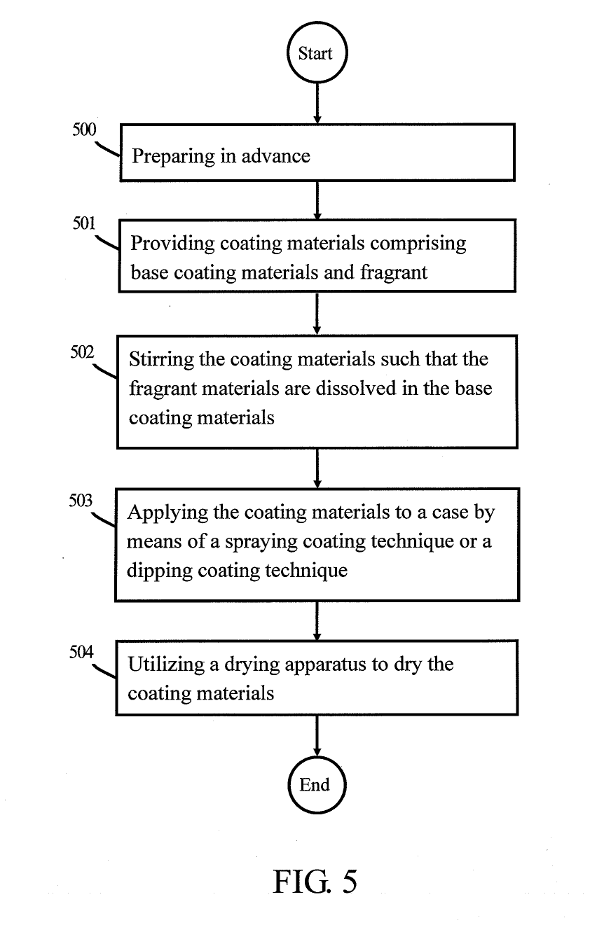

Please refer to both FIG. 2 and FIG. 5. FIG. 5 is a flowchart of a method for forming a coating layer on a case of the present invention, so as to provide a more practical process of the present invention:

Step 500: Preparing in advance.

The present invention cleans the surface of the cover 12 to get rid of oil or other impurities, so as to facilitate the application of coating materials to the cover 12.

Step 501: Providing coating materials.

The present invention provides the coating materials, wherein the coating materials comprise base coating materials and fragrant materials 18. The composition of the coating materials has been described above; therefore, there is no need for another detailed description.

Step 502: Stirring the coating materials.

The present invention stirs the coating materials, such that the fragrant materials 18 are evenly dissolved in the base coating materials.

Step 503: Applying the coating materials to a case by means of a spraying coating technique or a dipping coating technique.

### (1) Spraying Coating Technique:

If the coating materials are formed on the case 12 by means of the spraying coating technique, the spraying distance is between 20 and 30 centimeters. The spraying distance is defined as the distance between a spray gun and the case 12.

### (2) Dipping Coating Technique:

If the coating materials are formed on the case 12 by means of the dipping coating technique, the dipping speed is between 20 and 70 mm/min. The dipping speed is defined as the speed at which the case 12 moves throughout the process of being completely dipped into the coating materials and completely withdrawn from the coating materials.

Step 504: Utilizing a drying apparatus to dry the coating materials.

The drying apparatus can comprise a drying oven or a tube. If the drying apparatus is of the tube type, it can be a high pressure mercury lamp or a halogen mercury lamp. All procedures are completed after the coating materials completely dry out.

Although the present invention has been explained in relation to its preferred embodiments, it is to be understood that many other possible modifications and variations can be made without departing from the spirit and scope of the invention as hereinafter claimed.

What is claimed is:

1. A case with fragrance, comprising:
a cover; and
a coating layer formed on the cover, the coating layer comprising fragrant materials capable of releasing fragrance.

2. The case as claimed in claim 1, wherein the coating layer comprises a base layer, and the fragrant materials are spread in the base layer.

3. The case as claimed in claim 2, wherein the base layer is selected from the group consisting of metallic paint, epoxy resin, polyurethane resin, and UV-curing acrylic resin.

4. The case as claimed in claim 2, wherein the fragrant materials are in the form of capsules.

5. The case as claimed in claim 1, wherein the coating layer is formed by utilizing a spraying coating technique or a dipping coating technique to apply coating materials to the cover.

6. The case as claimed in claim 5, wherein the coating materials are in the form of liquid, and the fragrant materials are in the form of capsules or liquid.

7. The case as claimed in claim 1, wherein the coating layer is formed on the outside of the cover, such that the coating layer enhances the effect of releasing fragrance through contact with a user's hand.

8. The case as claimed in claim 1, wherein the cover is a cover of an electronic product.

9. The case as claimed in claim 1, wherein the material of the cover is selected from the group consisting of plastic, metal, leather, wood, and synthetic material.

10. The case as claimed in claim 1, wherein the fragrant materials release plant fragrance.

11. An electronic device, comprising:
a case with fragrance, the case comprising:
a cover; and
a coating layer formed on the cover, the coating layer comprising fragrant materials capable of releasing fragrance.

12. The electronic device as claimed in claim 11, wherein the coating layer comprises a base layer, and the fragrant materials are spread in the base layer.

13. The electronic device as claimed in claim 11, wherein the coating layer is formed by utilizing a spraying coating technique or a dipping coating technique to apply coating materials to the cover.

14. A method for forming a coating layer on a case, comprising the following steps:
providing coating materials, the coating materials comprising base coating materials and fragrant materials; and
applying the coating materials to the case.

15. The method as claimed in claim 14 further comprising:
stirring the coating materials, such that the fragrant materials are dissolved in the base coating materials.

16. The method as claimed in claim 14, wherein the base coating materials are selected from the group consisting of metallic paint, epoxy resin, polyurethane resin and, UV-curing acrylic resin.

17. The method as claimed in claim 14, wherein the fragrant materials are in the form of capsules.

18. The method as claimed in claim 14, wherein the coating materials are in the form of liquid, and the fragrant materials are in the form of capsules or liquid.

19. The method as claimed in claim 14, wherein in the step of applying the coating materials to the case, the coating materials are formed on the case by means of a spraying coating technique or a dipping coating technique.

20. The method as claimed in claim 19, wherein the coating materials are formed on the case by means of the spraying coating technique, wherein a spraying distance is between 20 and 30 centimeters.

21. The method as claimed in claim 19, wherein the coating materials are formed on the case by means of the dipping coating technique, wherein a dipping speed is between 20 and 70 mm/min.

22. The method as claimed in claim 19 further comprising:
utilizing a drying apparatus to dry the coating materials formed on the case.

23. The method as claimed in claim 22, wherein the drying apparatus comprises a drying oven or a tube.

24. The method as claimed in claim 23, wherein the tube is a high pressure mercury lamp or a halogen mercury lamp.
